(19) United States
(12) Patent Application Publication
(10) Pub. No.: US 2024/0316260 A1
(43) Pub. Date: Sep. 26, 2024

(54) CRADLE FOR AN INFUSION PUMP

(71) Applicant: B. Braun Medical Inc., Bethlehem, PA (US)

(72) Inventor: Benjamin Loomis, Topton, PA (US)

(73) Assignee: B. Braun Medical Inc., Bethlehem, PA (US)

(21) Appl. No.: 18/123,720

(22) Filed: Mar. 20, 2023

(51) Int. Cl. A61M 5/142; A61M 39/10

(52) U.S. Cl. CPC A61M 5/14244; A61M 39/10; A61M 2205/502

(57) ABSTRACT

A cradle securely mounts one or more infusion pumps to a pole without obstructing the display, buttons, or connector ports of the infusion pumps. A lower portion of the cradle includes connection pins sized/oriented to fit within respective connection holes of the infusion pump as well as an integrated strain relief mechanism for one or more cables. An upper portion of the cradle includes clamps for engaging a protrusion/ridge on a top surface of the infusion pumps. In a single cradle configuration, a recess within a back surface of the cradle mates with a pole clamp connector or a mounting bar in either a vertical or horizontal orientation. In a multiple cradle configuration, each cradle is secured indirectly to the pole clamp connector via a mounting bar and respective adapters. The mounting bar may be bent to introduce an offset between adjacent infusion pumps.

### FIELD OF THE INVENTION

The present disclosure is related to infusion pump apparatus and, more particularly, to cradles that, for example, provide integrated strain relief for one or more cables and that are sized and shaped to facilitate access to the display, buttons, and connector ports of mounted infusion pumps.

### BACKGROUND

Infusion pumps deliver controlled doses of fluids such as medications, analgesics, and nutrition to patients. Infusion pumps are particularly well suited to delivering controlled doses of fluids over long periods of time, e.g., several hours or days. While many infusion pumps are designed for bedside use, there are ambulatory versions available. Ambulatory infusion pumps allow a patient to move around while the infusion pump is in use.

Syringe pumps and peristaltic pumps are two conventional types of infusion pumps. A syringe pump depresses a cylinder within a syringe to deliver fluid from the syringe to a patient. A peristaltic pump acts on a tube to control the rate of fluid flow through the tube from a bottle or bag of fluid to a patient. Precise delivery of fluids is desirable to optimize treatment of a patient as there are many fluids where small variations can be critical.

To limit access to medications by unauthorized personnel, fluid medications administered by infusion pumps may be stored in an intravenous (IV) fluid hanger housing. Such housings may be mounted on an IV pole and used bedside in a hospital setting. While such housings provide safe operation without unauthorized access to the IV fluid medications, such housings do not accommodate ambulatory infusion pumps while providing access to the user interfaces of the ambulatory infusion pumps for programming by medical personnel while simultaneously preventing access to the fluid medications.

### SUMMARY

Examples described herein are directed to a cradle for securely mounting one or more infusion pumps to a pole without obstructing the display, buttons, or connector ports of the infusion pumps. During use of the infusion pumps, it is often desirable to securely position the infusion pumps in a room (e.g., on a designated pole or a rail of a hospital bed) in order to free the operator's hands for other tasks. The cradle assembly described herein allows the operator to connect one or more infusion pumps quickly and easily to a pole or a rail without covering the screen, buttons, or ports of the infusion pump.

In sample configurations, a “claw like” attachment mechanism of the cradle grips the infusion pump on the top and bottom so as to enable the infusion pump to be quickly and easily snapped into engagement with its mount without obscuring a front face or connector ports of the infusion pump. A lower portion of the cradle includes connection pins sized/oriented to fit within respective connection holes of the infusion pump. The lower portion of the cradle further includes an integrated strain relief mechanism for one or more cables. The integrated strain relief mechanism includes a pair of holes that are sized to receive cables connected to ports on the bottom of the infusion pump. An upper portion of the cradle includes clamps for engaging a protrusion/ridge on a top surface of the infusion pump.

In the sample configurations, a cradle for an infusion pump includes a lower portion having connection pins that are at least one of sized or oriented to fit within corresponding connection holes at a bottom of the infusion pump, and an upper portion having means for engaging a protrusion or ridge on a top of the infusion pump. A back surface of the cradle has a recess that is sized and shaped to mate with a clamp connector of a pole clamp in either a vertical orientation or a horizontal orientation whereby the infusion pump when mounted in the cradle may be attached to a pole without obstructing a display or connector ports of the infusion pump. The lower portion of the cradle may further include integrated strain relief means such as a pair of holes that are sized to receive at least one cable connected to the connector ports of the infusion pump.

The cradle may be included in a cradle assembly for mounting the infusion pump to a pole or a rail. The cradle assembly further includes a clamp connector adapted to connect to the pole or the rail and fasteners for connecting the cradle to the clamp connector. In a single cradle configuration, the cradle may be connected directly to the clamp connector by the fasteners or may be connected to a mounting bar that is disposed between the cradle and the clamp connector. The mounting bar may include a recess that is sized and shaped to mate with the clamp connector in either a vertical orientation or a horizontal orientation. An adapter may be disposed between the mounting bar and the cradle that adapted to fit within the recess of the back surface of the cradle in either a vertical orientation or a horizontal orientation. In a multiple cradle configuration, the mounting bar may be configured to connect the clamp connector to multiple cradles. The mounting bar may be bent to introduce an offset between adjacent infusion pumps when mounted so as to facilitate access to the sides of each infusion pump.

### BRIEF DESCRIPTION OF THE DRAWINGS

The drawing figures depict multiple views of one or more implementations, by way of example only, not by way of limitations. In the figures, like reference numerals refer to the same or similar elements. The same numeral is used to represent the same or similar element across the multiple views. If multiple elements of the same or similar type are present, a letter may be used to distinguish between the multiple elements. When the multiple elements are referred to collectively or a non-specific one of the multiple elements is being referenced, the letter designation may be dropped.

FIG. 1 is a perspective view of an example ambulatory peristaltic infusion pump.

FIG. 2 is a perspective view of an example cassette with a free flow prevention clamp for use with the ambulatory peristaltic infusion pump of FIG. 1.

FIG. 3 is a partial perspective view of the ambulatory peristaltic infusion pump of FIG. 1 illustrating cams that engage the free flow prevention clamp when the cassette is coupled to the ambulatory peristaltic infusion pump.

FIGS. 4 and 5 are cutaway views of the ambulatory peristaltic infusion pump of FIG. 1 illustrating pump sliders and cam rods for moving the pump sliders.

FIG. 6 is a front view of a lockbox that securely stores an ambulatory peristaltic infusion pump and a fluid bag in a sample configuration.

FIG. 7 is a front view of the lockbox of FIG. 6 without the ambulatory infusion pump or fluid bag in a sample configuration.

FIG. 8 is a top view of the lockbox of FIG. 6 including the ambulatory infusion pump and the fluid bag in a sample configuration.

FIG. 9 is a top view of the lockbox of FIG. 6 without the ambulatory infusion pump or the fluid bag in a sample configuration.

FIG. 10 is a front perspective view of the lockbox of FIG. 6 showing the cable access to the connector ports of the mounted ambulatory infusion pump in a sample configuration.

FIG. 11 is an exploded view illustrating the lockbox of FIG. 6 mounted to a pole via a lockable pole clamp in a sample configuration.

FIG. 12 is a front view of the cradle for securely mounting the ambulatory infusion pump to a pole or a rail in a sample configuration.

FIG. 13 is an enlarged view of the bottom portion of the cradle of FIG. 12 in a sample configuration.

FIG. 14 is an enlarged view of the top portion of the cradle of FIG. 12 in a sample configuration.

FIG. 15 is a perspective view of an ambulatory infusion pump mounted in the cradle of FIG. 12 in a sample configuration.

FIG. 16 is a bottom perspective view of the ambulatory infusion pump mounted in the cradle of FIG. 12, showing access to the connector ports in a sample configuration.

FIG. 17 illustrates a mounting bar for mounting the cradle to a pole clamp in a sample configuration.

FIG. 18 illustrates multiple cradles connected to a single pole clamp via a mounting bar in a sample configuration.

FIG. 19 illustrates an exploded back perspective view of the multiple cradles, mounting bar, and pole clamp of FIG. 18 in a sample configuration.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well-known methods, procedures, components, and circuitry have been described at a relatively high-level, without detail, in order to avoid unnecessarily obscuring aspects of the present teachings. Moreover, while described with respect to an ambulatory peristaltic infusion pump for pain management, homecare, outpatient infusions, and the like, it will be appreciated by those skilled in the art that the lockbox described herein may be used with a variety of other pump types.

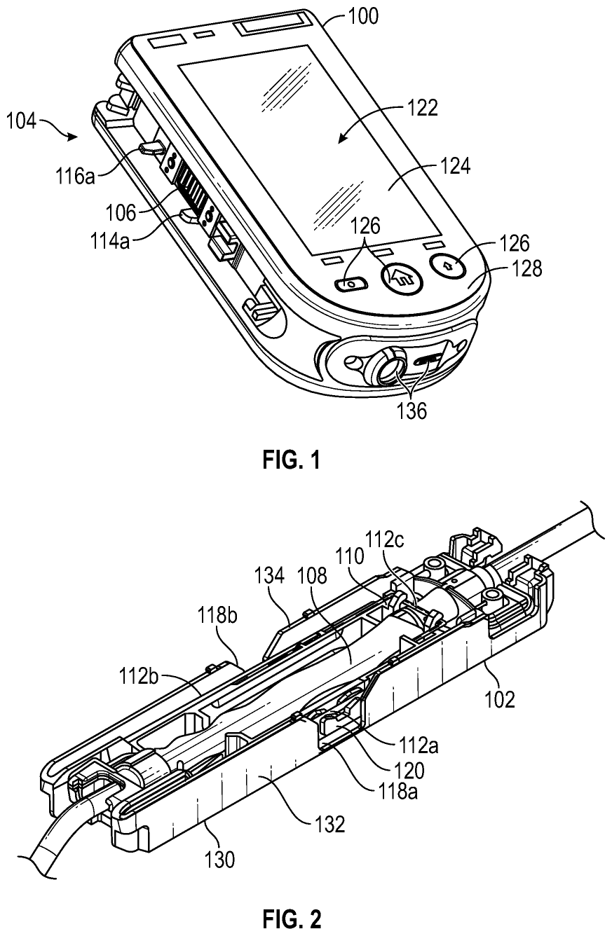

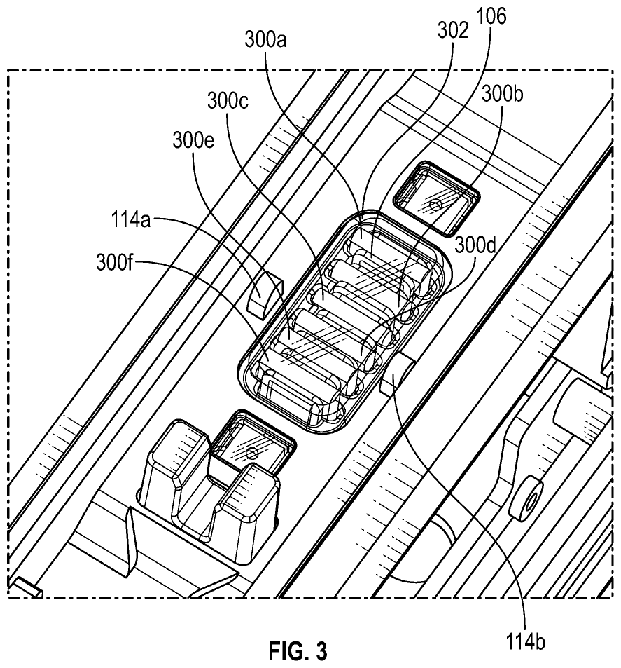

FIG. 1 depicts an example ambulatory peristaltic infusion pump 100, while FIG. 2 depicts an example cassette 102 for use with the ambulatory peristaltic infusion pump 100. The ambulatory peristaltic infusion pump 100 includes a receptacle 104 configured to receive the cassette 102. A peristaltic pump 106 within the receptacle 104 acts upon a tube 108 extending through a channel within the cassette 102 to pump fluid from a fluid container (e.g., a bag or a bottle; not shown) into a patient. An example free flow prevention clamp 110 is positioned within the cassette 102 to allow fluid flow through the tube 108 when the cassette 102 is coupled to the ambulatory peristaltic infusion pump 100 within the receptacle 104 (during which time the peristaltic pump 106 controls fluid flow through the tube 108) and to selectively cut off fluid flow through the tube 108 when the cassette 102 is not coupled to the ambulatory peristaltic infusion pump 100 in order to prevent unintentional fluid flow through the tube 108 (e.g., free flow).

The ambulatory peristaltic infusion pump 100 includes a user interface 122 for interacting with the ambulatory peristaltic infusion pump 100. The illustrated user interface 122 includes a display 124 (which may be a touchscreen) and buttons 126. A user controls the operation of the ambulatory peristaltic infusion pump 100 via the user interface 122. The ambulatory peristaltic infusion pump 100 additionally includes a housing 128 for containing and supporting the components of the ambulatory peristaltic infusion pump 100 such as the peristaltic pump 106, electronics, power supplies, and the like.

The free flow prevention clamp 110 includes a first elongate section 112a, a second elongate section 112b, and a clamping section 112c. The housing 130 of the cassette 102 supports the free flow prevention clamp 110. The clamping section 112c is positioned within the cassette geometry such that, when the cassette 102 is received within the receptacle 104 of the ambulatory peristaltic infusion pump 100, the clamping section 112c extends across the channel receiving the tube 108. The housing 130 of the cassette 102 may be rigid plastic or other material capable of supporting the tube 108 and free flow prevention clamp 110.

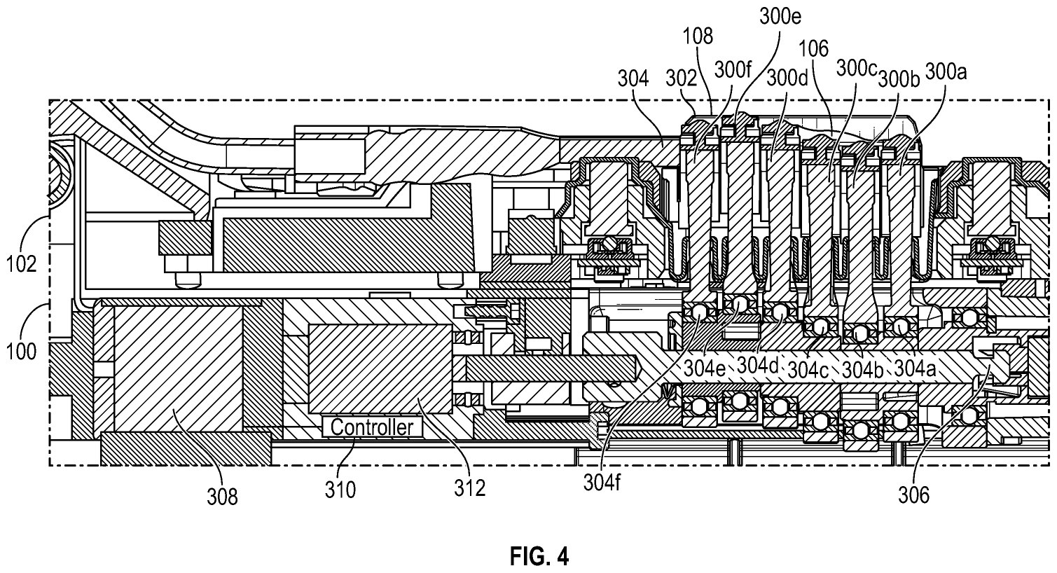

The ambulatory peristaltic infusion pump 100 also includes a pair of arc cams 114a and 114b (FIG. 3). First arc cam 114a is shown on one side of the receptacle illustrated in FIG. 1, but the second arc cam 114b is hidden from view. The pair of arc cams 114a and 114b engage the elongate sections 112a, 112b of the free flow prevention clamp 110 in order to lift the clamping section 112c. Additionally, the ambulatory pump 100 includes a pair of wedge cams 116a and 116b. A first wedge cam 116a is shown on one side of the receptacle 104 illustrated in FIG. 1, but the second wedge cam 116b is hidden from view. The pair of wedge cams 116a and 116b transition the free flow prevention clamp 110 from an open, manufactured/shipped state to an operational state, which is described in further detail below.

The cassette 102 also includes a first cutout 118a in a sidewall 132 of the cassette 102 and a second cutout 118b in an opposite sidewall 134 of the cassette 102. Additionally, the cassette 102 includes a touch pad 120 positioned on the first elongate section 112a adjacent a mid-point of the first elongate section 112a and the first cutout 118a. The touch pad 120 and cutout 118a together facilitate engagement of the first elongate section 112a by a finger of an operator in order to manually lift the clamping section 112c to allow fluid flow through the tube 108 (e.g., for priming the cassette 102) when the cassette 102 is not received within the receptacle 104 of the ambulatory peristaltic infusion pump 100. The touch pad 120 may be a press fit piece of rigid plastic. Although the touch pad 120 is illustrated as only on the first elongate section 112a, the touch pad 120 also may be provided on the second elongate section 112b.

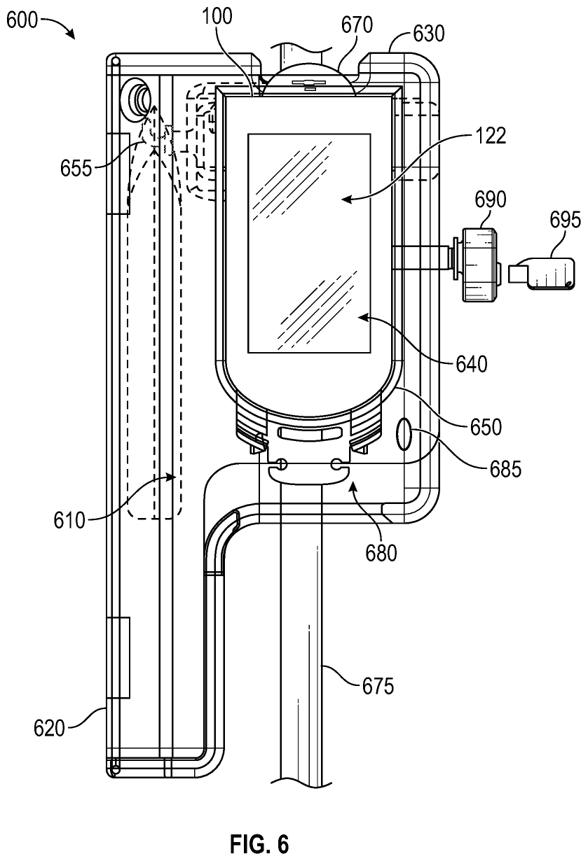

The ambulatory infusion pump 100 further includes connector ports 136 that provide electronic access for control and for powering the ambulatory infusion pump 100 when used in the configurations of FIGS. 6-19 described below.

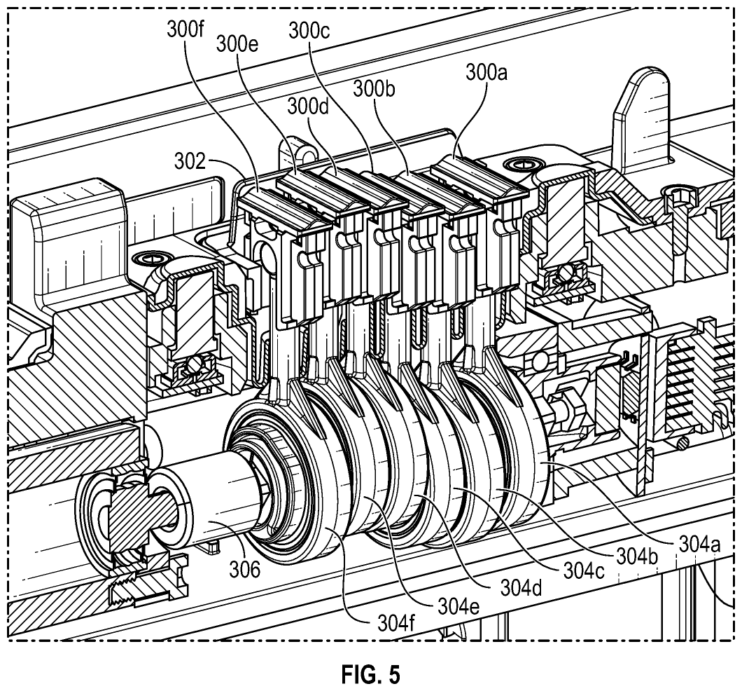

FIG. 3 depicts the arc cams 114a and 114b and peristaltic pump 106 of the ambulatory peristaltic infusion pump 100. The peristaltic pump 106 includes multiple pump fingers 300 (six pump fingers 300a-f illustrated in FIG. 3). A flexible barrier (seal) 302 (FIG. 4) separates the pump fingers 300 (and other pump components of a pumping mechanism) from the receptacle 104 receiving the cassette 102 with the tube 108. The flexible barrier 302 provides a barrier between the fluid delivery apparatus/cassette and the pumping mechanism to prevent fluid from damaging components of the pumping mechanism.

FIGS. 4 and 5 are cutaway views of the ambulatory peristaltic infusion pump 100 with the cassette 102 inserted into the receptacle 104 of the ambulatory infusion pump 100. Multiple cams 304 (six cams 304a-f) supported by a camshaft 306 act on respective pump sliders 300a-300f. The cams 304a-304f respectively raise and lower the pump sliders 300a-300f, which engage the tube 108 of the cassette 102 in order to force fluid though the tube 108. A pump motor 308 under control of a controller 310 turns the camshaft 306 by way of a gearbox 312. As the camshaft 306 turns, the cams 304a-300f, which are offset from each other in an axial direction, raise and lower respective pump sliders 300a-300f. For example, cam 304a raises and lowers pump slider 300a; cam 304b raises and lowers pump slider 300b, and the like. The controller 310 may be a standalone or embedded processor configured to carry out instructions in order to control operations of the ambulatory peristaltic infusion pump 100.

The controller 310 may include a main controller such as a dual core 32 bit processor from NXP of Eindhoven, Netherlands (e.g., model #MCIMX7S5EVM08SC), a microcontroller from NXP (e.g., model #MKV11Z128VLF7), a pump motor driver from ST Microelectronics of Geneva, Switzerland (e.g., model #STSPIN250), and a magnetic encoder from Austriamicrosystems of Premstaetten, Austria (e.g., model number AS5601-ASOM). The microcontroller receives pump camshaft revolutions per minute (RPM) corresponding to the infusion rate from a system control core of the main processor. The microcontroller develops a pulse width modulation (PWM) motor drive parameter relating to the desired camshaft RPM. The PWM output of the microcontroller becomes the motor drive input to the pump motor driver, which contains motor drive transistors and protection circuitry. The rotation of the camshaft 306 of the pumping mechanism is measured by the magnetic encoder. At specified time intervals, the output of the encoder is read by the microcontroller, which uses the encoder value to compute the speed of the camshaft 306 and the position of the pump rotation. These values are then used to modify the PWM output to maintain the correct camshaft RPM.

While specifically adapted for use outside of a bedside setting, the ambulatory infusion pump 100 may also be used in a bedside setting. In such a case, the ambulatory infusion pump 100 may be connected to tubing (e.g., tubing 108, FIG. 2) extending from a bag containing an infusate that is mounted on a bedside intravenous (IV) pole. However, in order to limit access to the infusate (which may be an opiate, for example), it is desired to further provide a lockbox that is adapted to securely house the bag containing the infusate as well as the ambulatory infusion pump 100.

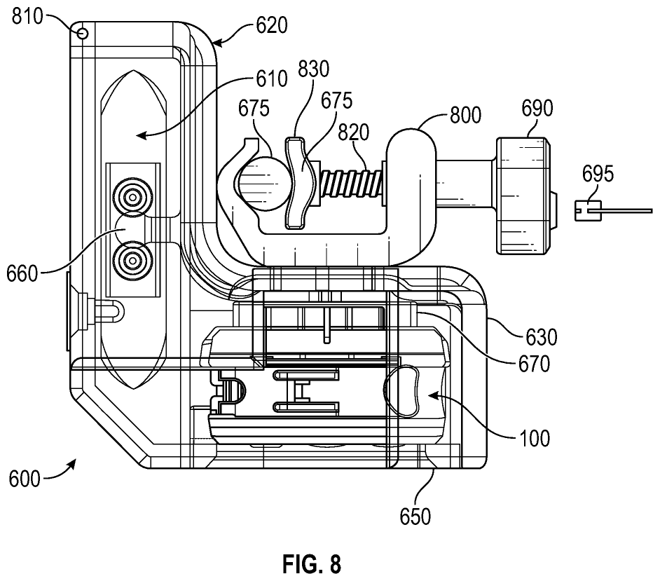

FIG. 6 is a front view of a lockbox 600 for securely storing the ambulatory infusion pump 100 and a fluid bag 610 in a sample configuration. As illustrated, the lockbox 600 has an L-shaped housing (from the top view, FIG. 8) including a first portion 620 adapted to securely store the fluid bag 610 and a second portion 630 adapted to securely store the ambulatory infusion pump 100 while enabling access to the display 124 via a front opening 640 in a front cover 650 of the lockbox 600. In a sample configuration, the first portion 620 and the second portion 630 are sized and shaped to distribute weight evenly when fluid bag 610 is received by a bag hook 655 in the first portion 620 and the ambulatory infusion pump 100 is received by the cradle 670 in the second portion 630. As will be apparent from FIG. 8, the L-shaped configuration of the lockbox 600 distributes weight around the circumference of the pole 675, when mounted, to provide better stability over conventional lockboxes.

As further illustrated in FIG. 6, the lockbox 600 includes a bottom opening 680 that provides access to the connector ports 136 on the bottom of the ambulatory infusion pump 100. The lockbox 600 also includes a lock 685 that locks the front cover 650 to the first portion 620 and the second portion 630 of the lockbox 600 once the ambulatory infusion pump 100 has been mounted in the cradle 670 and the fluid bag 610 has been hung from the bag hook 655. A screw lock 690 further locks a lockable pole clamp 800 (FIG. 8) to lock the lockbox 600 onto the pole 675. The screw lock 690 may be opened by a key 695 that is controlled by medical personnel.

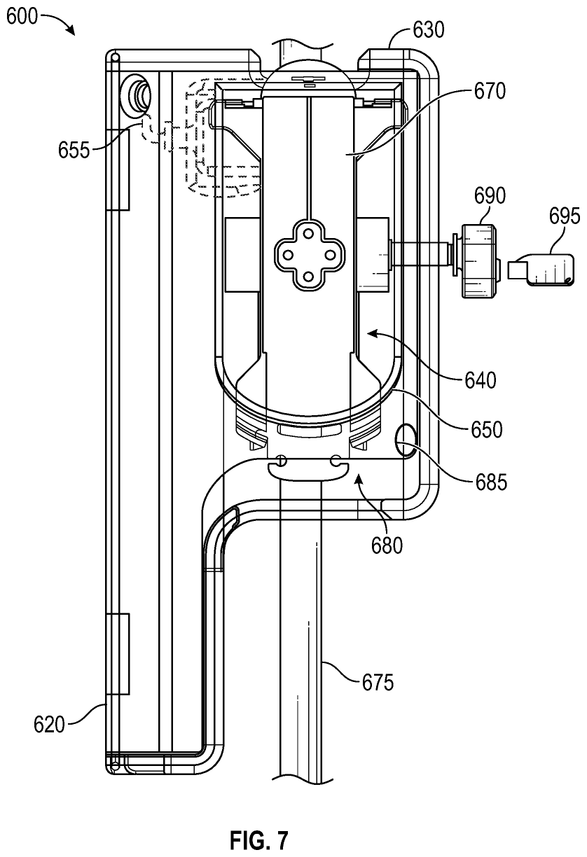

FIG. 7 is a front view of the lockbox 600 of FIG. 6 without the ambulatory infusion pump 100 or fluid bag 610 in a sample configuration. FIG. 7 better illustrates the cradle 670 that is adapted to hold the ambulatory infusion pump 100 to allow the display 124 to be viewed through the front opening 640 of the front cover 650 when closed and locked by lock 685.

The lockbox 600 thus includes a housing having first portion 620 and second portion 630 sized and configured to respectively receive a fluid bag 610 and an ambulatory infusion pump 100. The second portion 630 supports the cradle 670 for receiving the ambulatory infusion pump 100. The front opening 640 in the front cover 650 allows access to the ambulatory infusion pump's user interface 122 (e.g., touchscreen and buttons) when the ambulatory infusion pump 100 is mounted in the cradle 670 and the front cover 650 is closed. Bottom opening 680 allows access to electrical connector ports (e.g., for attaching a bolus cord). The lock 685 on the front cover 650 secures the front cover 650 to the first portion 620 and the second portion 630 to prevent access to the fluid bag 610 and fluid tubing 108 running from the fluid bag 610 to the ambulatory infusion pump 100 during operation of the ambulatory infusion pump 100.

FIG. 8 is a top view of the lockbox 600 of FIG. 6 including the ambulatory infusion pump 100 and the fluid bag 610. As illustrated in FIG. 8, the front cover 650 is coupled to the main support structure including first portion 620 and second portion 630 of the lockbox 600 via a hinge 810 that allows the front cover 650 to open. Also, once the screw 820 of the pole clamp 800 is rotated to secure the pole clamp 800 and lockbox 600 to the pole 675 with locking clamp knob 830, the screw 820 can be locked in place by screw lock 690 to prevent removal of the pole clamp 800 or lockbox 600 from the pole 675 without using the key 695.

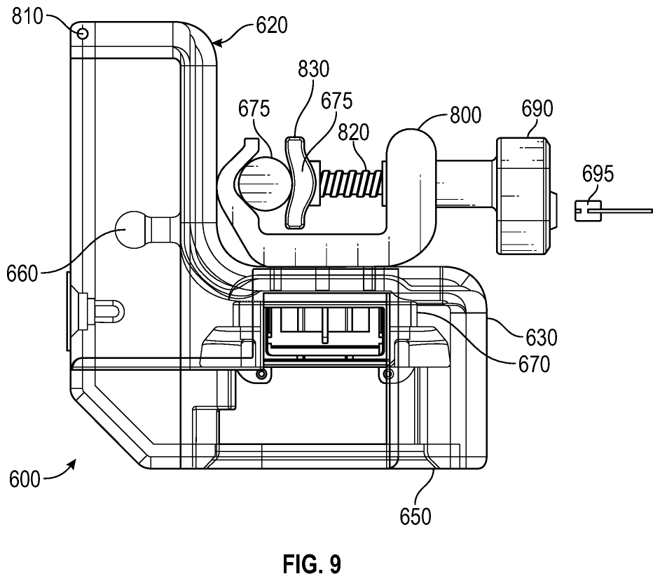

FIG. 9 is a top view of the lockbox 600 of FIG. 6 without the ambulatory infusion pump 100 or the fluid bag 610, better illustrating the cradle 670.

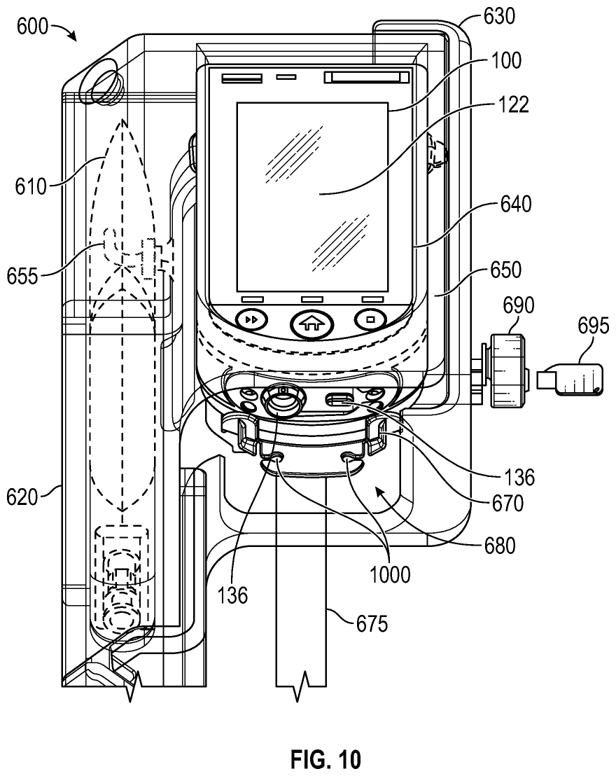

FIG. 10 is a front perspective view of the lockbox 600 of FIG. 6 showing the cable access to the connector ports 136 of the ambulatory infusion pump 100 via the bottom opening 680. As illustrated, the cradle 670 further includes integrated strain relief means in the form of cable restraints 1000 that prevent cables from being easily pulled out of the connector ports 136 during use.

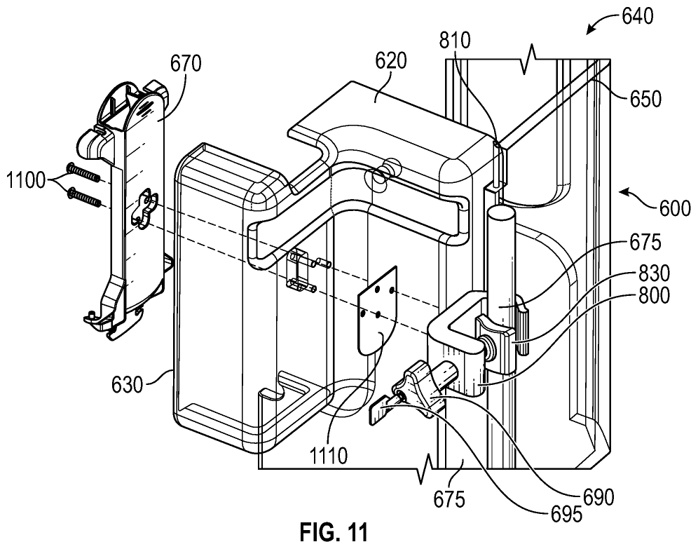

FIG. 11 is an exploded view illustrating the lockbox 600 of FIG. 6 mounted to pole 675 using the lockable pole clamp 800. As illustrated, the second portion 630 of the lockbox 600 is mounted to the lockable pole clamp 800 by screws 1100 that are inserted from the interior of the lockbox 600 through mounting holes in the cradle 670 and counterpart mounting holes in the second portion 630 of the lockbox 600 and that pass through a plate 1110 to terminate in the lockable pole clamp 800. As illustrated, the mounting holes in the cradle 670 and in the second portion 630 of the lockbox 600 may be selectively arranged horizontally or vertically for termination in the lockable pole clamp 800. It will be appreciated that this arrangement prevents access to the screws 1100 when the lockbox 600 is closed and locked, thereby preventing removal of the lockable pole clamp 800 from the lockbox 600.

The lockbox 600 described herein thus provides a support for an ambulatory infusion pump 100 for pumping fluid from a fluid bag 610 to a patient in a bedside configuration. The lockbox 600 limits access to the ambulatory infusion pump 100 and the infusate in the fluid bag 610 except by authorized medical personnel who have the key 695 to the screw lock 690 and/or a key to the lock 685. Also, the L-shaped configuration of the lockbox 600 distributes weight around the circumference of the pole 675, when mounted, to provide better stability while maintaining security.

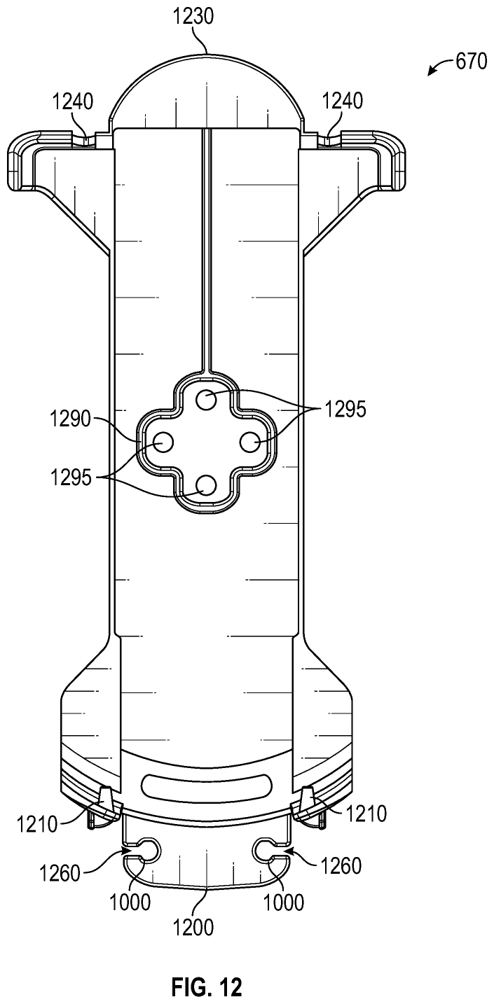

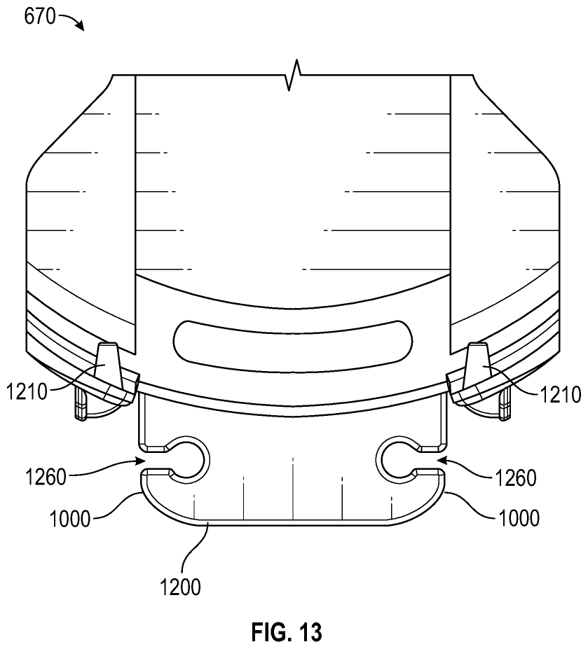

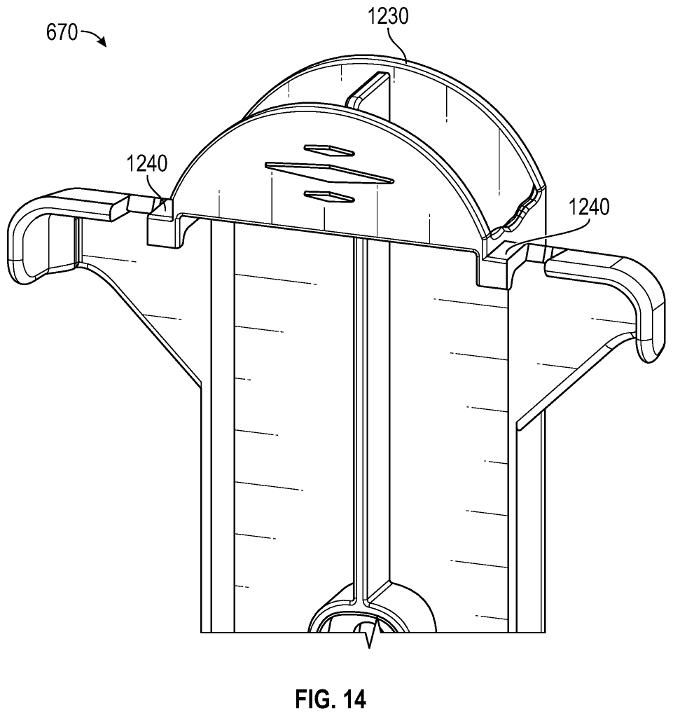

FIG. 12 is a front view of the cradle 670 for securely mounting the ambulatory infusion pump 100 to pole 675 or a rail without obstructing the user interface 122 or connector ports 136 in a sample configuration. FIG. 13 is an enlarged view of the bottom portion 1200 of the cradle of FIG. 12, while FIG. 14 is an enlarged view of the top portion 1230 of the cradle of FIG. 12 in a sample configuration. As illustrated, the cradle 670 is adapted to hold the ambulatory infusion pump 100 in a “claw like” attachment mechanism that grips the ambulatory infusion pump 100 on the top and bottom to enable the ambulatory infusion pump 100 to be quickly and easily snapped into engagement with its mount without obscuring the user interface 122 or connector ports 136 of the ambulatory infusion pump 100.

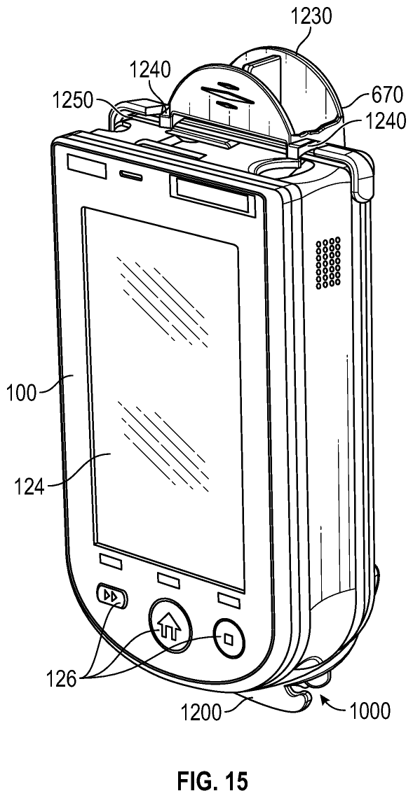

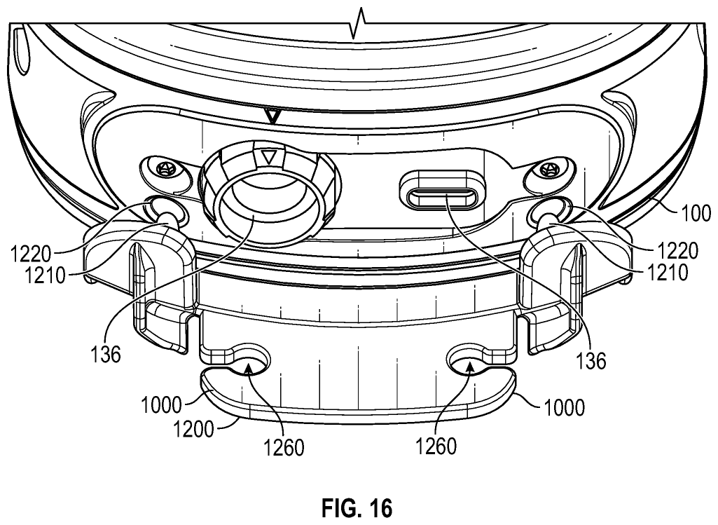

As illustrated in FIG. 12, the lower portion 1200 of the cradle 670 includes connection pins 1210 that are sized and/or oriented to fit within respective connection holes 1220 (FIG. 16) at the bottom of the ambulatory infusion pump 100. The upper portion 1230 of the cradle 670 includes engagement means in the form of clamps/clasps 1240 for engaging a protrusion/ridge 1250 (FIG. 15) on the top surface of the ambulatory infusion pump 100. To secure the ambulatory infusion pump 100 within the cradle 670, the connection pins 1210 on the lower portion 1200 of the cradle 670 are first inserted into the connection holes 1220 of the ambulatory infusion pump 100. The top portion of the ambulatory infusion pump 100 is then pressed into engagement with the upper portion 1230 of the cradle 670 until the connection clamps/clasps 1240 of the upper portion 1230 of the cradle 670 snap fit over the protrusion/ridge 1250 on the top surface of the ambulatory infusion pump 100. To remove the ambulatory infusion pump 100 from the cradle 670, the upper portion 1230 of the cradle 670 is pushed to release the connection clamps/clasps 1240 from the protrusion/ridge 1250 on the top surface of the ambulatory infusion pump 100. The ambulatory infusion pump 100 is then lifted off of the connection pins 1210 on the lower portion 1200 of the cradle 670. This arrangement allows an operator to connect or disconnect an ambulatory infusion pump 100 quickly and easily to or from the cradle 670 without covering the display 124, buttons 126, or connector ports 136 of the ambulatory infusion pump 100.

As also illustrated in FIG. 12, the lower portion 1200 of the cradle 670 further includes an integrated strain relief means 1000 to provide strain relief for one or more cables. The integrated strain relief means 1000 includes a pair of holes 1260 that are sized to receive cables connected to the connector ports 136 on the bottom of the ambulatory infusion pump 100.

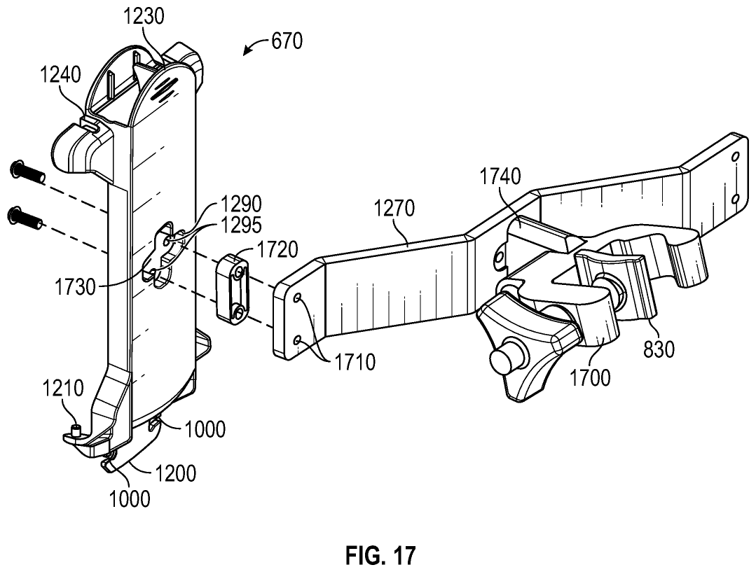

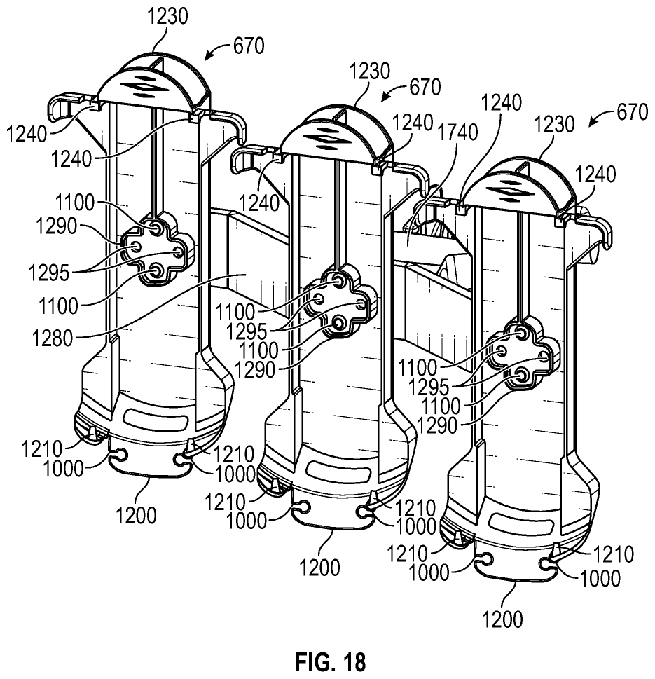

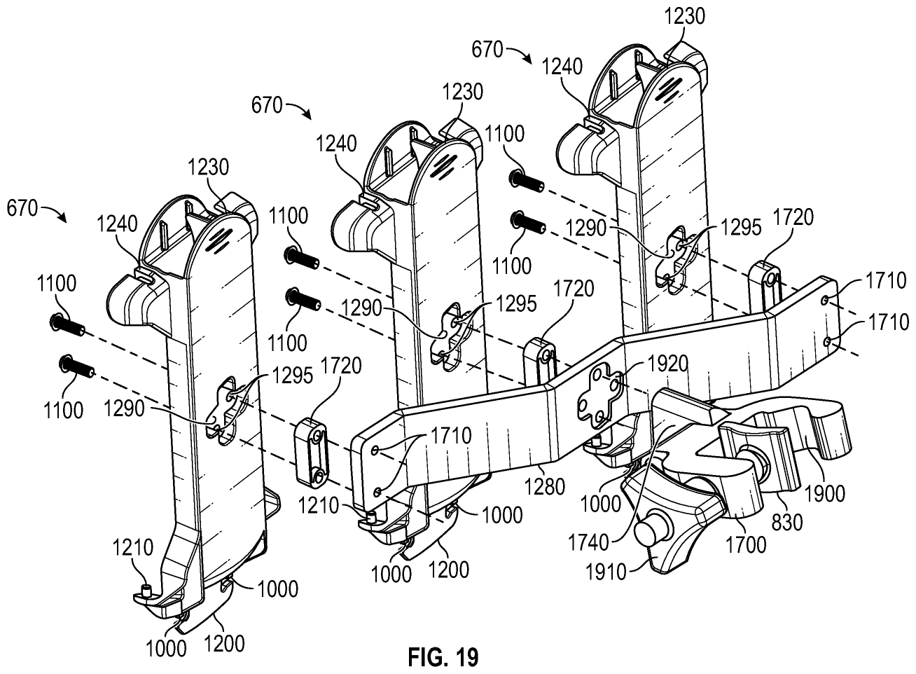

In sample configurations, the cradle 670 may be coupled directly to a pole clamp (e.g., lockable pole clamp 800) or to a pole clamp 1700 via a mounting bar 1270 (FIG. 17) or mounting bar 1280 (FIGS. 18-19) that enable a single pole clamp to be used with one or more cradles 670 as depicted in FIGS. 17-19. The cradle 670 may include a clamp orientation guide 1290 in the form of a recess 1730 (FIG. 17) including vertically oriented and horizontally oriented holes 1295 for use in mounting the cradle 670 to a pole clamp or a mounting bar 1270 or 1280 using any combination of the vertically and horizontally oriented holes 1295.

FIG. 15 is a perspective view of an ambulatory infusion pump 100 mounted in the cradle 670 of FIG. 12 in a sample configuration. As illustrated, the display 124 and buttons 126 remain accessible when the ambulatory infusion pump 100 is snapped into the cradle 670 by snap fitting the connection clamps/clasps 1240 of the upper portion 1230 of the cradle 670 over the protrusion/ridge 1250 on the top surface of the ambulatory infusion pump 100. As shown in FIG. 16, the connector ports 136 of the ambulatory infusion pump 100 also remain accessible when the ambulatory infusion pump 100 is mounted in the cradle 670.

The cradle 670 can be used in a single cradle implementation and in a multi-cradle implementations.

In a single cradle implementation, the cradle 670 may be mounted directly to a lockable pole clamp 800 (with or without a lockbox 600) as illustrated in FIG. 11. Alternatively, the cradle 670 may be mounted to a mounting bar 1270 that is, in turn, mounted to a pole clamp 1700 as illustrated in FIG. 17. The clamp orientation guide 1290 of the cradle 670 may be used to orient the holes 1295 of the clamp orientation guide 1290 with corresponding holes 1710 of the mounting bar 1270. An adapter 1720 may be used to space the cradle 670 from the mounting bar 1270. The adapter 1720 may be adapted to fit snugly into the clamp orientation guide 1290 to prevent rotation of the cradle 670 once mounted. The clamp orientation guide 1290 may include a recess 1730 within a back surface of the cradle 670 that is sized and shaped to mate with adapter 1720, the lockable pole clamp 800, and/or a clamp connector 1740 of the pole clamp 1700 in either a vertical orientation or a horizontal orientation.

Thus, in the single cradle implementation, each cradle 670 includes a clamp orientation guide 1290 within the back surface of the cradle 670 that is sized and shaped to mate with a clamp connector 1740 of the pole clamp 800 or 1700 in either a vertical orientation (with respect to a pole 675 as shown) or a horizontal orientation. This allows the pole clamp 800 or 1700 to be used with a single cradle with or without the use of the mounting bar 1270 or adapter 1720. For example, the cradle 670 may be secured directly to the pole clamp 800 or 1700 with fasteners (e.g., screws) 1100 or to the mounting bar 1270 via adapter 1720 and fasteners 1100.

FIGS. 18 and 19 illustrate multiple cradles connected to a single pole clamp 1700 via a mounting bar 1280. As illustrated, each cradle 670 is secured indirectly to the clamp connector 1740 of the pole clamp 1700 with fasteners 1100 via a mounting bar 1280 and respective adapters 1720. As illustrated in FIG. 19, the pole clamp 1700 may include a pole receiving area 1900 and a pole securing mechanism 1910 in addition to the clamp connector 1740.

In the multi-cradle implementation, the mounting bar 1280 enables mounting of multiple ambulatory infusion pumps 100 via multiple cradles 670 using a single pole clamp 1700. The illustrated mounting bar 1280 is configured to receive three ambulatory infusion pumps 100 and cradles 670 and is bent to introduce an offset between adjacent ambulatory infusion pumps 100 to, for example, facilitate access to the sides of the ambulatory infusion pumps 100. The mounting bar 1280 may include a bar orientation guide 1920 in the form of a recess that is sized and shaped to mate with the clamp connector 1740 of the pole clamp 1700 as well as the adapter 1720 in either a vertical orientation (with respect to a pole 675 within the pole receiving area 1900) or a horizontal orientation (e.g., to facilitate connection to a bed rail). In this implementation, each cradle 670 is secured indirectly to the clamp connector 1740 of the pole clamp 1700 with fasteners (e.g., screws) 1100 via the mounting bar 1280 and respective adapters 1720 that are sized and shaped to mate with the clamp orientation guide 1290 of the cradle 670 and the bar orientation guide 1920 of the mounting bar 1280. It will be appreciated that a clamp orientation guide 1290 also may be provided on the mounting bar 1270 for mounting a single ambulatory infusion pump 100.

The cradle 670 described herein thus enables secure attachment of one or more ambulatory infusion pumps 100 to a pole 675 or a rail without obstructing the display 124, buttons 126, or connector ports 136 of the ambulatory infusion pump 100. One or more cradles 670 also may be readily adapted for inclusion in one or more lockbox assemblies to limit undesirable access to the ambulatory infusion pump(s) 100 when in use.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is ordinary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 105 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” “includes,” “including,” or any other variation thereof, are intended to cover a non-exclusive inclusion, such that a process, method, article, or apparatus that includes a list of elements does not include only those elements but may include other elements not expressly listed or inherent to such process, method, article, or apparatus. An element preceded by “a” or “an” does not, without further constraints, preclude the existence of additional identical elements in the process, method, article, or apparatus that includes the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

In addition, in the foregoing Detailed Description, it can be seen that various features are grouped together in various examples for the purpose of streamlining the disclosure. This method of disclosure is not to be interpreted as reflecting an intention that the claimed examples require more features than are expressly recited in each claim. Rather, as the following claims reflect, the subject matter to be protected lies in less than all features of any single disclosed example. Thus, the following claims are hereby incorporated into the Detailed Description, with each claim standing on its own as a separately claimed subject matter.

While the foregoing describes what is considered to be the best mode and other examples, it is understood that various modifications may be made and that the subject matter disclosed herein may be implemented in various forms and examples, and that they may be applied in numerous applications, only some of which have been described herein. It is intended by the following claims to claim any and all modifications and variations that fall within the true scope of the present concepts.

What is claimed is:

1. A cradle for an infusion pump, comprising:
a lower portion having connection pins that are at least one of sized or oriented to fit within corresponding connection holes at a bottom of the infusion pump;
an upper portion having means for engaging a protrusion or ridge on a top of the infusion pump; and
a back surface having a recess that is sized and shaped to mate with a clamp connector of a pole clamp in either a vertical orientation or a horizontal orientation,
wherein the infusion pump when mounted in the cradle may be attached to a pole or a rail without obstructing a display or connector ports of the infusion pump.

2. The cradle of claim 1, wherein the lower portion further comprises integrated strain relief means for accepting at least one cable.

3. The cradle of claim 2, wherein the integrated strain relief means comprises a pair of holes that are sized to receive the at least one cable connected to the connector ports of the infusion pump.

4. A cradle assembly for mounting an infusion pump to a pole or a rail, comprising:
a cradle comprising a lower portion having connection pins that are at least one of sized or oriented to fit within corresponding connection holes at a bottom of the infusion pump, an upper portion having means for engaging a protrusion or ridge on a top of the infusion pump, and a back surface having a recess;
a clamp connector adapted to connect to the pole or the rail; and
fasteners for connecting the cradle to the clamp connector,
wherein the recess is sized and shaped to mate with the clamp connector in either a vertical orientation or a horizontal orientation, and
wherein the infusion pump when mounted in the cradle may be attached to the pole or the rail without obstructing a display or connector ports of the infusion pump.

5. The cradle assembly of claim 4, further comprising a mounting bar that is disposed between the cradle and the clamp connector.

6. The cradle assembly of claim 5, wherein the mounting bar comprises a mounting bar recess that is sized and shaped to mate with the clamp connector in either the vertical orientation or the horizontal orientation.

7. The cradle assembly of claim 5, further comprising an adapter disposed between the mounting bar and the cradle, the adapter adapted to fit within the recess of the back surface of the cradle in either the vertical orientation or the horizontal orientation.

8. The cradle assembly of claim 5, wherein the mounting bar is configured to connect the clamp connector to multiple cradles.

9. The cradle assembly of claim 8, wherein the mounting bar comprises a mounting bar recess that is sized and shaped to mate with the clamp connector in either the vertical orientation or the horizontal orientation.

10. The cradle assembly of claim 8, further comprising adapters disposed between the mounting bar and each cradle, each adapter adapted to fit within the recess of the back surface of each cradle in either the vertical orientation or the horizontal orientation.

11. The cradle of claim 8, wherein the mounting bar is bent to introduce an offset between adjacent infusion pumps when mounted.
